(19) United States
(12) Patent Application Publication
(10) Pub. No.: US 2022/0095957 A1
(43) Pub. Date: Mar. 31, 2022

(54) Estimating Caloric Expenditure Based on Center of Mass Motion and Heart Rate

(71) Applicant: Apple Inc., Cupertino, CA (US)

(72) Inventors: Di Wu, San Jose, CA (US); Hui Chen, Los Altos, CA (US); Paige N. Stanley, San Jose, CA (US); James Ochs, San Francisco, CA (US)

(21) Appl. No.: 17/033,621

(22) Filed: Sep. 25, 2020

(51) Int. Cl. A61B 5/11; A61B 5/00; A61B 5/024

(52) U.S. Cl. CPC A61B 5/1122; A61B 5/681; A61B 5/4866; A61B 2562/0219; A61B 5/742; A61B 5/0004; A61B 5/0022; A61B 5/02405

(57) ABSTRACT

Embodiments are disclosed for estimating caloric expenditure based on center of mass motion and heart rate. In an embodiment, a method comprises: obtaining acceleration and rotation rate of a wearable device worn on a limb of a user; transforming the acceleration and rotation rate into an inertial frame; determining a vertical component of acceleration, rotation rate magnitude and vertical component of rotational acceleration due to limb rotation; determining a work rate (WR) based caloric expenditure based on the vertical component of acceleration, rotation rate magnitude and a correlation coefficient that measures a correlation between the vertical component of acceleration and the vertical component of rotational acceleration; obtaining heart rate (HR) data from a heart rate sensor of the wearable device; determining an HR based caloric expenditure based on the HR data; and fusing, the WR based caloric expenditure with the HR based caloric expenditure to get a fused caloric expenditure.

### TECHNICAL FIELD

This disclosure relates generally to activity monitoring using wearable devices.

### BACKGROUND

The metabolic equivalent of task (MET) is defined as a ratio of the rate of energy expended by an individual during physical activity to the rate of energy expended by the user at rest (referred to as the resting metabolic rate (RMR)). Many studies have shown that the conventional 1-MET baseline overestimates actual resting oxygen consumption and energy expenditures by about 20% to 30% on average. Therefore, an accurate calculation of MET for a specific individual requires data specific to the user and the activity.

Modern wearable devices (e.g., smart watches, fitness bands) are often used by individuals during fitness activities to determine their caloric expenditure during the fitness activity. Some wearable devices include inertial sensors (e.g., accelerometers, angular rate sensors) that are used to estimate a work rate (WR) based MET for the user wearing the device. Some wearable devices include a heart rate (HR) sensor that provides HR data that can be used with user estimated VO₂ MAX (maximal oxygen consumption) and other data (e.g., users weight, age) to estimate HR based MET.

For certain physical activities, such as dancing, the motion detected at the user's wrist (e.g., measured by smart watch or fitness band) may not be correlated with motion at the user's hip, i.e., core body motion. Studies have shown, however, that more calories are expended by motion at the hip (hereinafter, referred to as “center of mass motion”) than are expended by motion at the wrist. For example, while dancing the user may move their arms while their body core is stationary. This type of physical activity can result in the estimated number of calories expended by the user being larger than the actual number of calories expended by the user during the physical activity based on motion detected at the wrist of the user.

### SUMMARY

Embodiments are disclosed for estimating caloric expenditure based on center of mass motion and heart rate.

In an embodiment, a method comprises: obtaining, using one or more processors, acceleration and rotation rate in a body frame of a wearable device worn on a limb of a user engaged in a physical activity; transforming, using the one or more processors, the acceleration and rotation rate into an inertial frame; determining, using the one or more processors, vertical acceleration in the inertial frame, rotation rate magnitude and vertical acceleration in the inertial frame due to the user's limb rotation; determining, using the one or more processors, a work rate (WR) based caloric expenditure for the user based on the vertical acceleration, rotation rate magnitude and a correlation coefficient that measures a correlation between the vertical acceleration in the inertial frame and the vertical acceleration in inertial frame due to the user's limb rotation; obtaining, using the one or more processors, heart rate (HR) data from a heart rate sensor of the wearable device; determining, using the one or more processors, an HR based caloric expenditure based on the HR data; and fusing, the WR based caloric expenditure with the HR based caloric expenditure to get a fused caloric expenditure.

In an embodiment, the method further comprises: displaying, on a display of the wearable device the fused caloric expenditure, or sending by the wearable device the fused caloric expenditure to another device, or storing on wearable device or another device, the fused caloric expenditure.

In an embodiment, the method further comprises: obtaining, using the one or more processors, at least one of the user's weight and the user's height; and determining, using a WR model, the WR based caloric expenditure for the user based on the vertical acceleration, rotation rate magnitude, a correlation coefficient that measures a correlation between the vertical acceleration in the inertial frame and the vertical acceleration in inertial frame due to the user's limb rotation, the user's weight and the user's height.

In an embodiment, the method further comprises: compensating the HR data with a HR drift factor that is computed based on an amount of time the user is engaged in a physical activity of a specified intensity.

In an embodiment, the method further comprises: obtaining, using the one or more processors, the user's age and the user's estimated maximal oxygen uptake; and determining, using the one or more processors, an HR based caloric expenditure based on the HR data, the user's age and the user's estimated maximal oxygen uptake.

In an embodiment, fusing, the WR based caloric expenditure with the HR based caloric expenditure to get a fused caloric expenditure, further comprises: averaging the WR based caloric expenditure and the HR based caloric expenditure.

In an embodiment, the physical activity is dancing.

In an embodiment, the method further comprises: obtaining a plurality of reference vertical acceleration data points in the inertial frame that are associated with human body motion for a particular physical activity; subtracting the reference vertical acceleration from the vertical acceleration in the inertial frame that was measured at the user's wrist to obtain vertical acceleration in the inertial frame due to rotation of the user's limb; and determining a correlation coefficient representing the correlation using a linear regression model where the independent variable is the vertical acceleration in the inertial frame due to rotation of the user's limb and the dependent variable is the rotation rate of the user's limb in the inertial frame.

In an embodiment, the limb is the user's arm and the wearable device is worn on the user's wrist.

In an embodiment, the WR and HR caloric expenditures are based on metabolic equivalent of tasks (MET) values.

In an embodiment, a system comprises: motion sensors; one or more processors; memory storing instructions that when executed by the one or more processors, cause the one or more processors to perform operations comprising: obtaining, from the motion sensors, acceleration and rotation rate in a body frame of a wearable device worn on a limb of a user engaged in a physical activity; transforming the acceleration and rotation rate into an inertial frame; determining vertical acceleration in the inertial frame, rotation rate magnitude and vertical acceleration in the inertial frame due to the user's limb rotation; determining a work rate (WR) based caloric expenditure for the user based on the vertical acceleration, rotation rate magnitude and a correlation coefficient that measures a correlation between the vertical acceleration in the inertial frame and the vertical acceleration in inertial frame due to the user's limb rotation; obtaining heart rate (HR) data from a heart rate sensor of the wearable device; determining an HR based caloric expenditure based on the HR data; and fusing, the WR based caloric expenditure with the HR based caloric expenditure to get a fused caloric expenditure.

In an embodiment, the system further comprises: a display configured to display the fused caloric expenditure; and a wireless transmitter configured to send the fused caloric expenditure to another device.

In an embodiment, the operations further comprise: obtaining at least one of the user's weight and the user's height; and determining, using a WR model, the WR based caloric expenditure for the user based on the vertical acceleration, rotation rate magnitude, a correlation coefficient that measures a correlation between the vertical acceleration in the inertial frame and the vertical acceleration in inertial frame due to the user's limb rotation, the user's weight and the user's height.

In an embodiment, the operations further comprise: compensating the HR data with a HR drift factor that is computed based on an amount of time the user is engaged in a physical activity of a specified intensity.

In an embodiment, the operations further comprise: obtaining the user's age and the user's estimated maximal oxygen uptake; and determining an HR based caloric expenditure based on the HR data, the user's age and the user's estimated maximal oxygen uptake.

In an embodiment, the operations further comprise averaging the WR based caloric expenditure and the HR based caloric expenditure.

In an embodiment, the physical activity is dancing.

In an embodiment, the operations further comprise: obtaining a plurality of reference vertical acceleration data points in the inertial frame that are associated with human body motion for a particular physical activity; subtracting the reference vertical acceleration from the vertical acceleration in the inertial frame that was measured at the user's wrist to obtain vertical acceleration in the inertial frame due to rotation of the user's limb; and determining a correlation coefficient representing the correlation using a linear regression model where the independent variable is the vertical acceleration in the inertial frame due to rotation of the user's limb and the dependent variable is the rotation rate of the user's limb in the inertial frame.

In an embodiment, the limb is the user's arm and the wearable device is worn on the user's wrist.

In an embodiment, the WR and HR caloric expenditures are based on metabolic equivalent of tasks (MET) values.

Other embodiments can include an apparatus, computing device and non-transitory, computer-readable storage medium.

The details of one or more implementations of the subject matter are set forth in the accompanying drawings and the description below. Other features, aspects and advantages of the subject matter will become apparent from the description, the drawings and the claims.

### BRIEF DESCRIPTION OF THE DRAWINGS

FIG. 1 illustrates a system for determining caloric expenditure of a user based on center of mass motion and heart rate, according to an embodiment.

FIG. 2 is a scatter plot illustrating the correlation between motion at the wrist of a user and motion at the user's hip, according to an embodiment.

FIG. 3A is a scatter plot showing the correlation between a reference acceleration at the user's hip and vertical acceleration at the user's wrist, according to an embodiment.

FIG. 3B is a scatter plot showing the correlation of reference MET and vertical acceleration at the user's wrist, according to an embodiment.

FIG. 4 are plots illustrating examples of rotation rate when the user is dancing with leg only motion, arm only motion, or leg and arm together, according to an embodiment.

FIG. 5A illustrates the physics of rotational velocity about an elbow based on the rotation rate, according to an embodiment.

FIG. 5B are plots of vertical acceleration and rotational acceleration at the wrist, and a reference vertical acceleration at the hip for arm motion only, according to an embodiment.

FIG. 5C are plots of vertical acceleration and rotational acceleration at the wrist, and a reference vertical acceleration at the hip for arm plus leg motion, according to an embodiment.

FIG. 6A is a scatter plot illustrating the correlation between acceleration and rotation rate, according to an embodiment.

FIG. 6B is a scatter plot illustrating the correlation between work rate MET and reference METs, according to an embodiment.

FIG. 7 is a flow diagram of a process for computing a HR drift correction factor, according to an embodiment.

FIG. 8 illustrates the performance of a HR drift correction model, according to an embodiment.

FIG. 9 is a flow diagram of an example process estimating caloric expenditure based on center of mass motion and HR, according to an embodiment.

FIG. 10 is example wearable device architecture for a wearable device implementing the features and operations described in reference to FIGS. 1-9.

### DETAILED DESCRIPTION

### Example System

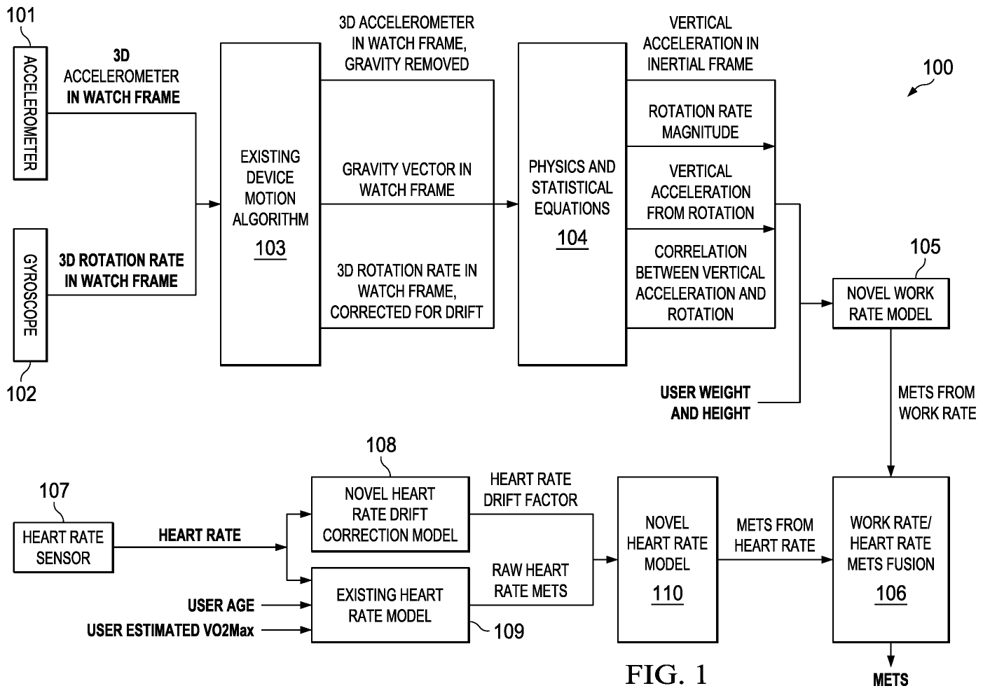

FIG. 1 illustrates a system 100 for determining caloric expenditure of a user based on center of mass motion and heart rate, according to an embodiment. System 100 can be implemented in wearable device, such as a smartwatch or fitness band worn on the wrist of a user during a physical activity. System 200 can also be worn on other limbs (e.g., one or both legs, both arms). Some examples of physical activity include but are not limited to: dancing (e.g., Bollywood, Zumba, Hip-Hop), aerobic exercises, tennis or any exercise or physical activity where the user's arms may be moving while their body core (torso) remains mostly stationary.

In an embodiment, the wearable device includes motion sensors, such as accelerometer 101 (e.g., a 3-axis MEMS accelerometer) and angular rate sensor 102 (e.g., 3-axis MEMS gyro) that provide a three-dimensional (3D) acceleration vector and 3D rotation rate vector in the wearable device body frame, respectively. The 3D acceleration and rotation rate vectors are input into motion processor 103, which computes and outputs a 3D acceleration vector in wearable device body coordinates with gravity removed, a gravity vector in the wearable device body frame and a 3D rotation rate vector in the wearable device body frame that is compensated for drift.

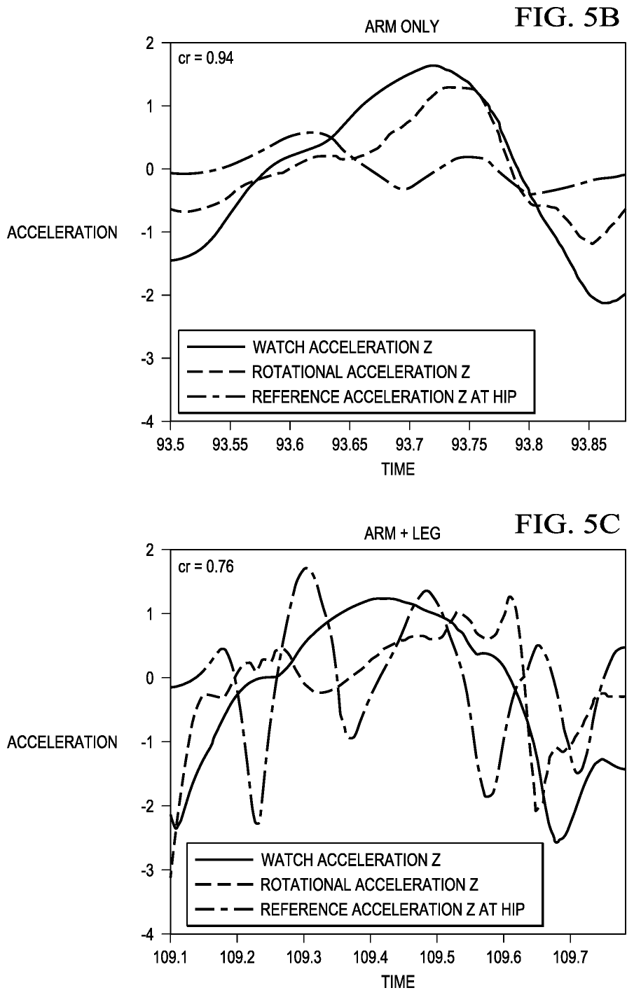

The output of motion processor 103 is input into physics/statistics processor 104, which computes and outputs vertical acceleration in an inertial world coordinate frame, a rotation rate magnitude, a vertical acceleration vector due to the rotation of the user's limb and a correlation coefficient indicating the correlation between the vertical acceleration and the rotation. In an embodiment, the physics/statistics processor 103 uses a coordinate transformation (e.g., a direction cosine matrix or quaternion) that uses yaw, pitch and roll angles derived by integrating the 3D rotation rate to transform the 3D acceleration vector (with gravity removed) from the wearable device body frame to the inertial frame, where the vertical component of inertial acceleration is normal to the Earth surface. The rotation magnitude is computed by taking the absolute value of the rotation rate vector. Vertical acceleration from rotation is determined as described in reference to FIG. 5A. In an embodiment, the correlation coefficient is the correlation of observed inertial vertical acceleration and its rotational component.

The inertial vertical acceleration, rotation rate magnitude, vertical acceleration due to limb rotation and the correlation coefficient are input into work rate model 105. In an embodiment, the user's weight and height are also input into work rate model 105, which computes WR MET values. In an embodiment, WR model 104 fuses the inertial vertical acceleration vector and the vertical acceleration estimated from the rotation rate vector using the correlation coefficient to provide an estimate of the user's center of mass motion (hereinafter also referred to as “body motion”), which is the motion of the user's body core. In an embodiment, the user's 3D body motion is computed by subtracting the arm only motion from the observed motion output by the sensors 101, 102. The body motion power could be described as shown in Equation [1]:

P{body motion}=P{observed motion}+P{−arm only motion}+2 Cov{observed motion,−arm only motion}, [1]

where P{ } indicates a power of the signal and Cov indicates a covariance.

The user's body motion is equal to the observed motion (e.g., from sensor data) minus arm only motion. The power/energy of the body motion can be expressed in the form of Equation [1]. It is assumed that the power/energy in the body motion signal is a good indicator for the body motion intensity. Equation [1] suggests that the observed vertical motion magnitude (observed inertial acceleration Accel Z), estimated arm only motion vertical magnitude (estimated from rotation as shown in FIG. 5A) and the correlation between these two signals are useful variables to predict calories burned. In an embodiment, the WR MET values are computed as a function of the energy expended by the user's estimated body motion, i.e., WR MET=f(P{body motion}, user's weight, user's height). The function f(.) can be any suitable WR MET formulation. In an embodiment, linear regression is used to fit a line to empirical data collected for various ranges of body motion energy expenditure for a particular physical activity (e.g., dancing), which is then calibrated or corrected by factors associated with the user's weight and height.

In an embodiment, HR sensor 107 measures the user's HR, which is input into a HR drift correction model 108 and HR model 109. In an embodiment, the HR sensor is embedded in the wearable device and comprises a number of light emitting diodes (LEDs) paired with photodiodes that can sense light. The LEDs emit light toward a user's body part (e.g., the user's wrist), and the photodiodes measure the reflected light. The difference between the sourced and reflected light is the amount of light absorbed by the user's body. Accordingly, the user's heart beat modulates the reflected light, which can be processed to determine the user's HR. The measured HR can be averaged over time to determine an average HR, which input into models 108, 109.

In an embodiment, HR model 109 estimates caloric expenditure during steady-state cardiovascular exercise using a relationship between heart rate and a maximal user estimated oxygen uptake (VO₂ MAX). During steady-state aerobic exercise, oxygen is utilized at a relatively consistent rate depending on the intensity of the exercise. There is an observable and reproducible relationship between heart rate and oxygen uptake. When workload intensity increases, heart rate increases and vice versa. If the user's resting heart rate, maximum heart rate, maximum oxygen uptake and weight are known, caloric expenditure can be estimated based on a percentage of their maximum heart rate or a percentage of their heart rate reserve.

In an embodiment, HR model 109 takes as input calibrated VO₂ MAX and the user's HR. The calibrated VO₂ MAX can be computed using the WR MET values (which is an implicit estimate of VO₂) with the user's measured HR data. The HR drift factor and HR MET values output by HR model 109 are input into HR rate model compensator 110, where the HR MET values are compensated for HR rate drift. In an embodiment, the HR drift factor is determined by the user's heart rate level in the past N minutes (e.g., N=12), and the time of being in moderate workout. The HR drift factor is applied to compensate raw HR MET values from the HR model according to Equation [3]. The output of HR rate model compensator 110 is the final HR MET values which are input together with the WR MET values into WR/HR Fusion processor 106. In an embodiment, WR/HR fusion processor 106 fuses the WR MET and HR MET values by, for example, averaging the WR MET and HR MET values.

The fused WR and HR MET values can be used for any desired purpose, including but not limited to: presentation on a display of the wearable device by a fitness or other application as an estimate of caloric expenditure, sent to another device for display and/or further processing by one or more other applications, stored on the wearable device for subsequent display/processing by one or more applications, or sent to a network-based computer system (the “cloud”), where the fused MET values can be displayed/processed by other devices connected to the network-based computer system.

In an embodiment, WR and HR calorie expenditure values (MET values) are estimated over successive, non-overlapping intervals of time referred to herein as “epochs.” An “epoch” can be x seconds (e.g., 2.56 seconds, corresponding to 256 samples of sensor data sampled at 100 Hz). Over each epoch, a single average MET value can be computed as described above, which is the best estimate of the energy expended by the user due to body motion. In another embodiment, arbitration logic compares the magnitude of the WR MET and HR MET values, and a confidence score associated with each of those MET values, and system 100 outputs only the WR MET or HR MET based on the confidence scores.

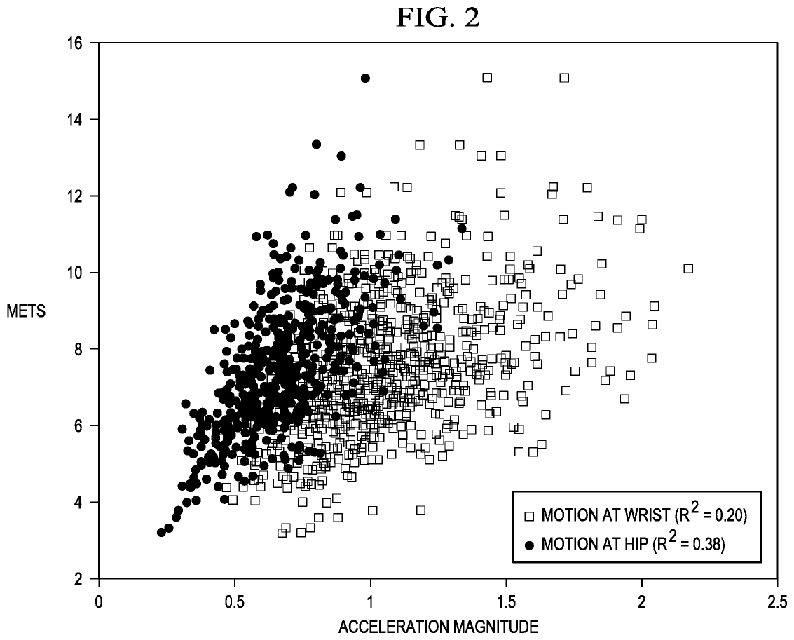

FIG. 2 is a scatter plot illustrating the correlation between MET values and acceleration magnitude for motion at the wrist and motion at the hip or body motion, according to an embodiment. As can be observed in FIG. 2, the acceleration measured at the user's hip is more correlated with METs than acceleration measured at the user's wrist. For this example data set, the R-squared (R²) value is 0.20 for wrist motion and 0.38 for hip motion. R² evaluates the scatter of the data points around a fitted regression line (not shown). For the same data set, a higher R² value represents smaller differences between the observed data and the fitted values. R² is represented as the percentage of the dependent variable variation that the liner model describes.

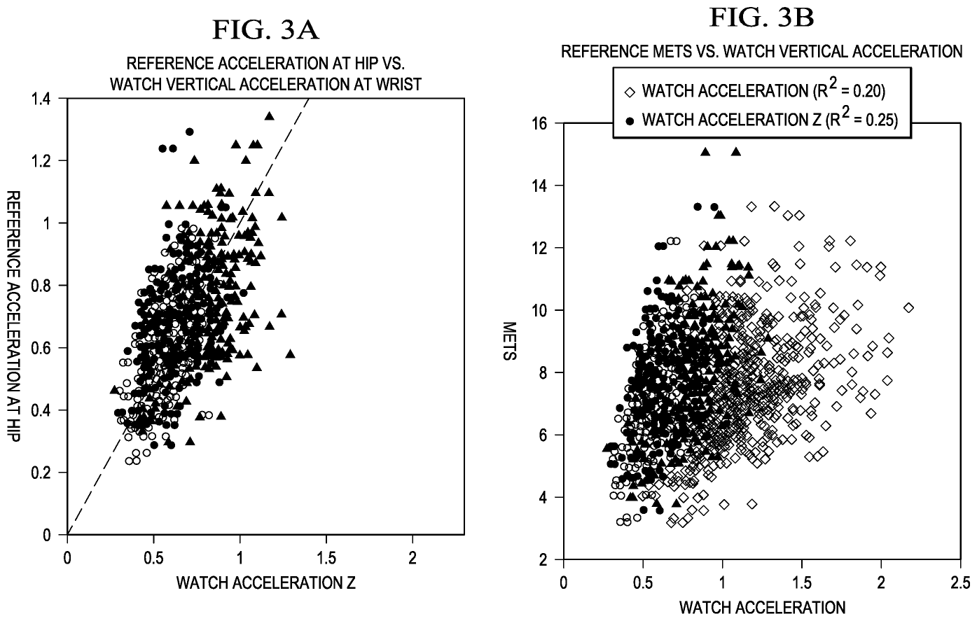

FIGS. 3A and 3B are scatter plots showing the correlation between a reference acceleration at the user's hip and vertical acceleration at the user's wrist, and the correlation of reference MET and vertical acceleration at the user's wrist, respectively, according to an embodiment. FIG. 3A shows a strong correlation between acceleration at the hip (i.e., body motion) and vertical acceleration at the wrist (“Accel Z”). FIG. 3B shows a strong correlation between caloric expenditure (METs) and the vertical acceleration at the wrist (R²=0.25), and a weaker correlation between 3D acceleration at the wrist (R²=0.20).

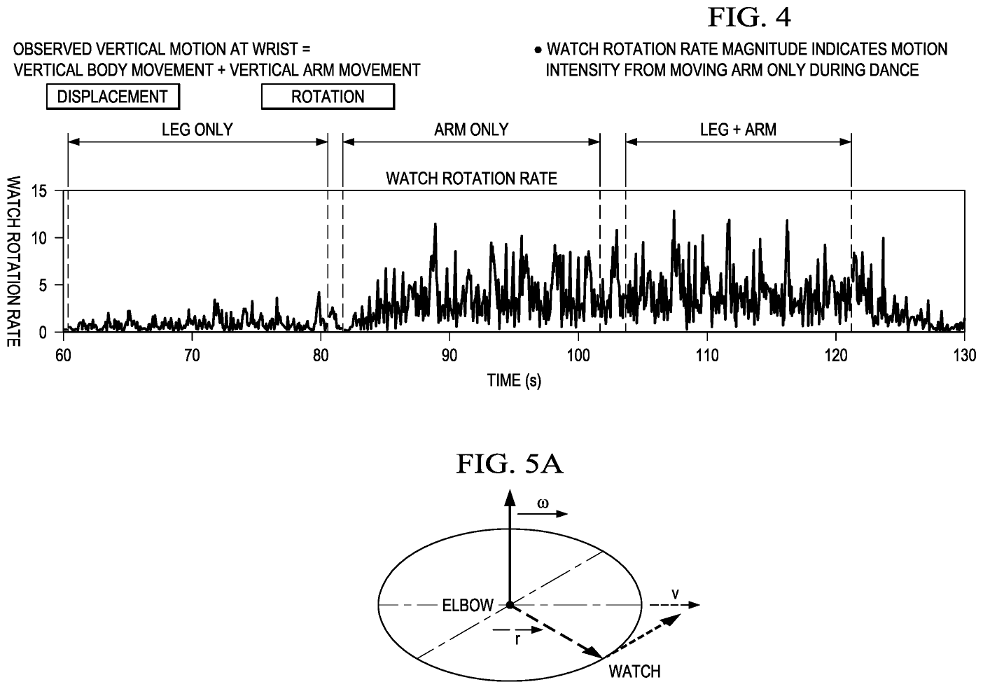

FIG. 4 are plots illustrating examples of rotation rate when the user is dancing with leg only motion, arm only motion, or leg and arm together, according to an embodiment. The observed vertical acceleration at the wrist is equal to the sum of the vertical body movement (vertical displacement) and the vertical arm movement due to rotation. The first plot is rotation rate for body (leg) motion, the second plot is of rotation rate due solely to arm rotation (i.e., magnitude of rotation rate output by 3-axis MEMs gyro) and the third plot shows a combination of body (leg) motion and arm motion. From these plots, it is clear that the rotation rate magnitude indicates motion intensity from arm only motion during the physical activity, which in this example is dancing. Note that the rotation rate in the body (leg) only plot is very small in magnitude compared to the arm only rotation rate.

FIG. 5A illustrates the physics of rotational velocity about an elbow based on the rotation rate, according to an embodiment. In FIG. 5A, the rotation is around the user's elbow and the resulting rotation velocity is computed according to Equation [2]:

\vec{V}ᵣₒₜₐₜᵢₒₙₐₗ=\vec{w}×\vec{r}, [2]

where \vec{w} is the rotation rate vector and \vec{r} is a vector from the wearable device to the elbow in the wearable device body frame. For a smart watch, \vec{r} represents the orientation of the watch's digital crown in the watch body frame.

FIG. 5B are plots of inertial vertical acceleration, inertial vertical acceleration due to arm rotation computed according to Equation [2] and a reference inertial vertical acceleration measured at the hip. The correlation coefficient (cr) between the inertial vertical acceleration at the wrist and the vertical acceleration due to rotation of the arm is 0.94, indicating a high correlation between these accelerations in arm only motion.

FIG. 5C are plots of inertial vertical acceleration, inertial vertical acceleration due to arm rotation, inertial vertical acceleration at the hip and a reference inertial vertical acceleration measured at the hip. The correlation (cr) between the inertial vertical acceleration at the wrist and the vertical acceleration due to rotation of the arm is 0.76, indicating less correlation due to the larger contribution of the body (leg) motion to the observed acceleration at the wrist.

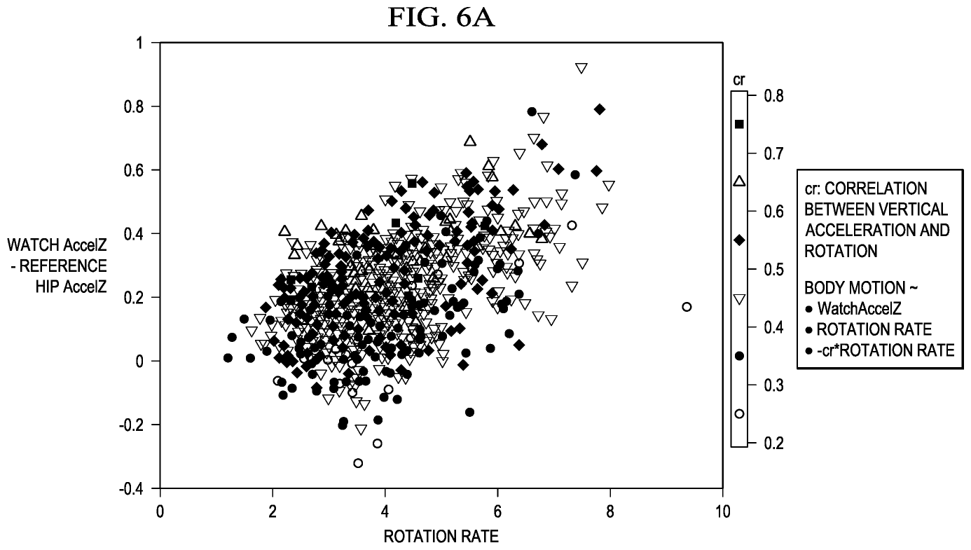

FIG. 6A is a scatter plot illustrating the correlation between acceleration and rotation rate, according to an embodiment. The left side independent axis is the magnitude of vertical acceleration from arm only motion. This plot indicates rotation rate is a good indicator for the magnitude of arm only motion. The correlation between the inertial vertical acceleration and the inertial vertical acceleration due to arm rotation could further explain the estimation error. Therefore, inertial vertical acceleration, rotation rate and the correlation coefficient can be used to predict the body motion magnitude.

FIG. 6B is a scatter plot illustrating the correlation between work rate MET and reference METs, according to an embodiment. As can be observed from the plot, the WR MET values computed using system 100 are strongly correlated with the reference MET values. Here, the reference MET values were computed using a metabolic cart (MET Cart) that essentially measures the oxygen consumed and the carbon dioxide produced by the user and then calculates (using the modified Weir equation) the energy expenditure for the patient, where the Weir equation is energy expenditure (EE)=(3.94× VO₂)+(1.1× (VCO₂))

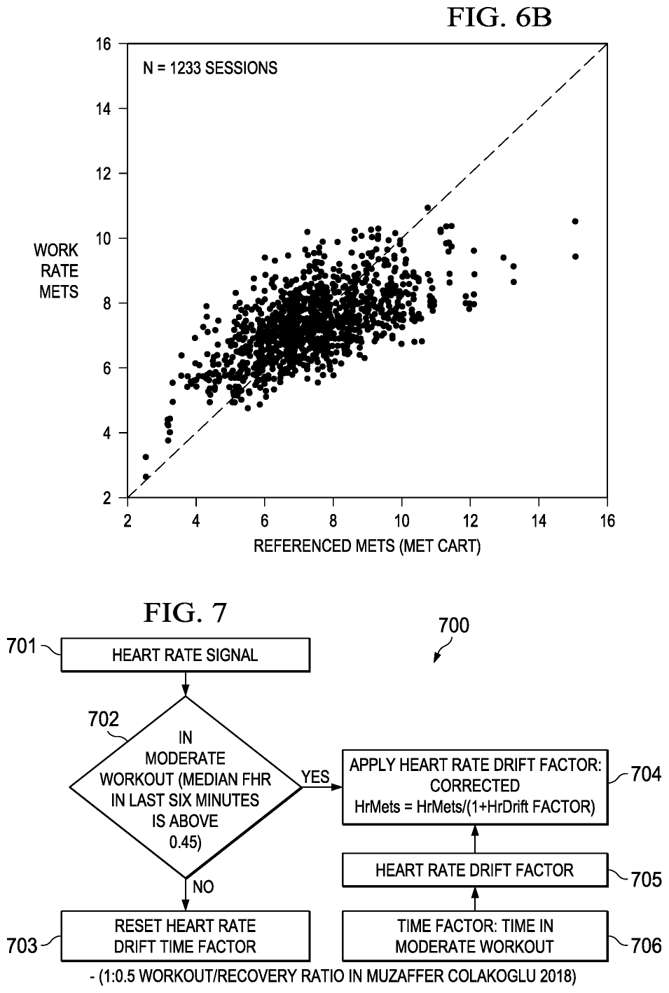

FIG. 7 is a flow diagram of a process 700 for computing HR drift factor, according to an embodiment. Studies have shown that during a workout the user's HR will change due to fatigue. This fatigue factor can result in large overestimates of HR. HR drift correction model 108 detects when the user has exceeded a fatigue HR threshold.

Process 700 starts with HR signal 701 output by HR sensor 107 being compared to a threshold value indicating that the user is engaged in a “moderate” workout (702). Here, a “moderate” workout is where the user has a medium HR for the last N minutes (e.g., 6 min) that is above a specified threshold K. In accordance with the user having a medium HR for the last N minutes that is above a specified threshold K, the HR drift factor 705 is applied to the HR data 704. Otherwise, the HR drift time is reset 703.

In an embodiment, HR drift factor 705 (HRDriftFactor) is based on a time factor 706 which is based on the amount of time the user has been engaged in the “moderate” workout. The time factor 706 can be obtained from a table of time factors associated with different workout times. The HR drift factor 705 is applied 704 to the HR MET values (HRMET) according to Equation [3]:

HRMET=HRMET/(1+HRDriftFactor). [3]

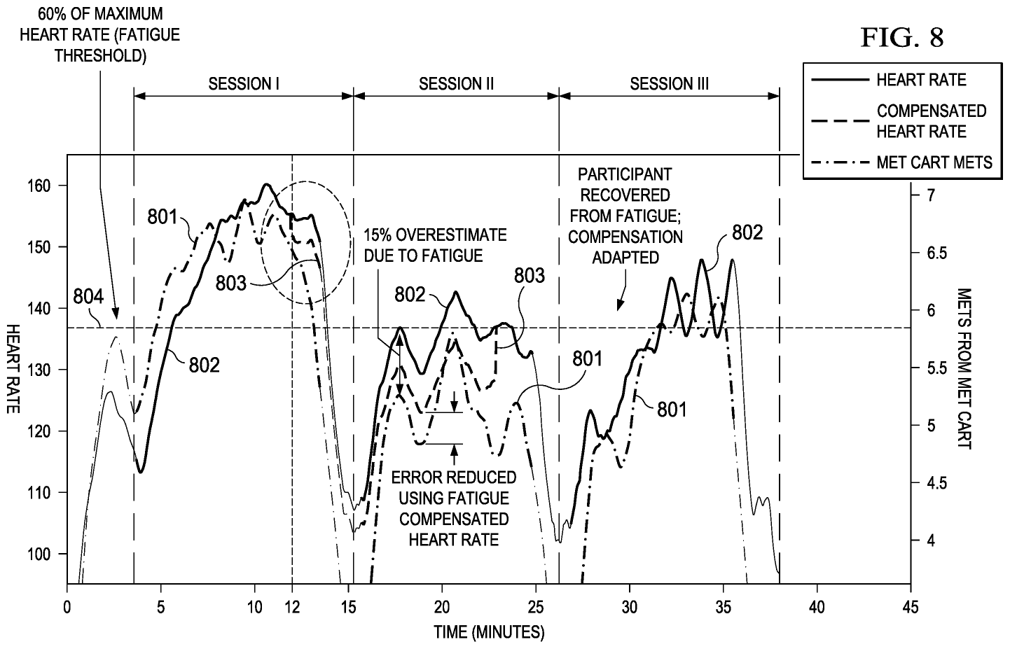

FIG. 8 illustrates the performance of a HR drift correction model shown in FIG. 7, according to an embodiment. In the example workout shown, there are three sections that start/end approximately at 0-15 mins, 15-26 mins and 26-38 mins. Plot 801 is MET Cart METS (truth data), plot 802 is HR data from HR sensor 107 and plot 803 is compensated HR data compensated by applying the HRDriftFactor shown in Equation [3]. Fatigue threshold 804 defines when the user has reached 60% of their maximum HR, which in this example is about 138 beats per minute (BPM).

In section I, at around 12 mins, the HR data is compensated for fatigue. In section II, one can observe that the uncompensated HR is overestimated by 15% compared to the MET Cart data due to fatigue of the user. When compensation is applied, however, the error is reduced. In section III, the user recovers (e.g., rehydrates, stops moving) and the compensation is adapted.

### Example Process

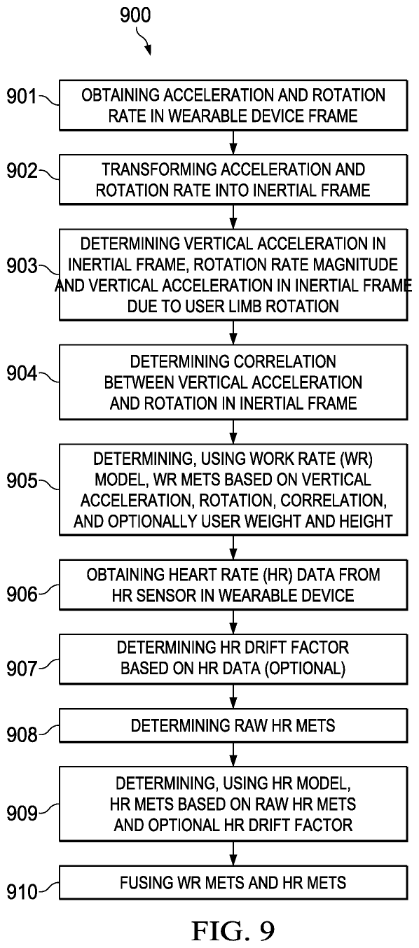

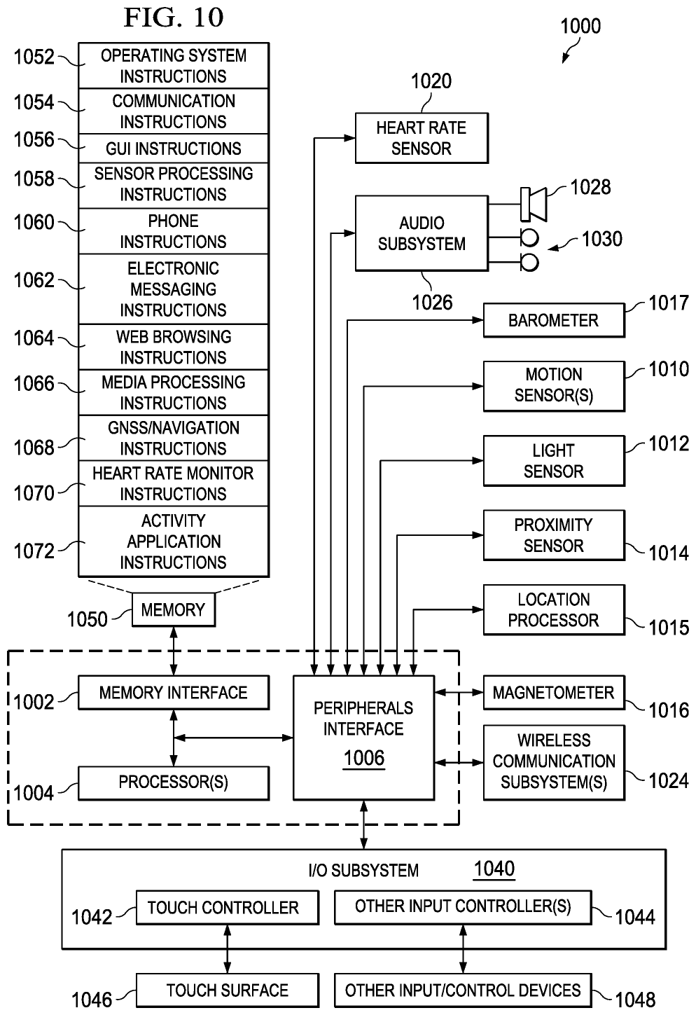

FIG. 9 is a flow diagram of an example process 900, according to an embodiment. Process 900 can be implemented using the wearable device architecture 1000 disclosed in reference to FIG. 10.

Process 900 includes obtaining acceleration and rotation rate in wearable device body frame (901), transforming the acceleration and rotation rate into inertial world reference frame (902), determining vertical acceleration in inertial frame, rotation rate magnitude and vertical acceleration in inertial frame due to arm rotation (903), determining correlation between the vertical acceleration in the inertial frame and the vertical acceleration in the inertial frame due to arm rotation (904) and determining, using a WR model, WR MET values based on the vertical acceleration in the inertial frame, vertical acceleration in the inertial frame due to arm rotation and a correlation coefficient representing the correlation between the two accelerations. In an embodiment, the user's weight and height can be used as correction factors by the WR model. Each of these steps were previously described in reference to FIGS. 1-8.

Process 900 continues by obtaining HR data from a HR rate sensor embedded in or coupled to the wearable device (906), determining HR drift factor based on the HR data (907), determining raw HR MET values using a first HR model based on user estimated VO₂ MAX and user age (908), determining, using a second HR model using the HR MET Values output by the first HR model and the HR drift factor, HR MET values (909) and fusing the WR MET values and the HR MET values to get final MET value (910). The user estimated VO₂ MAX can be computed from WR MET that was calibrated by the HR data and the user age can be entered by the user through, for example, a fitness application on the wearable device or a companion device coupled to the wearable device (e.g., a smart phone).

### Exemplary Wearable Computer Architecture

FIG. 10 illustrates example wearable device architecture 1000 implementing the features and operations described in reference to FIGS. 1-9. Architecture 1000 can include memory interface 1002, one or more hardware data processors, image processors and/or processors 1004 and peripherals interface 1006. Memory interface 1002, one or more processors 1004 and/or peripherals interface 1006 can be separate components or can be integrated in one or more integrated circuits.

Sensors, devices and subsystems can be coupled to peripherals interface 1006 to provide multiple functionalities. For example, one or more motion sensors 1010, light sensor 1012 and proximity sensor 1014 can be coupled to peripherals interface 1006 to facilitate motion sensing (e.g., acceleration, rotation rates), lighting and proximity functions of the wearable device. Location processor 1015 can be connected to peripherals interface 1006 to provide geo-positioning. In some implementations, location processor 1015 can be a GNSS receiver, such as the Global Positioning System (GPS) receiver. Electronic magnetometer 1016 (e.g., an integrated circuit chip) can also be connected to peripherals interface 1006 to provide data that can be used to determine the direction of magnetic North. Electronic magnetometer 1016 can provide data to an electronic compass application. Motion sensor(s) 1010 can include one or more accelerometers and/or gyros configured to determine change of speed and direction of movement of the wearable device. Barometer 1017 can be configured to measure atmospheric pressure around the mobile device.

Heart rate monitoring subsystem 1020 for measuring the heartbeat of a user who is wearing the device on their wrist. In an embodiment, subsystem 1020 includes LEDs paired with photodiodes for measuring the amount of light reflected from the wrist (not absorbed by the wrist) to detect a heartbeat.

Communication functions can be facilitated through wireless communication subsystems 1024, which can include radio frequency (RF) receivers and transmitters (or transceivers) and/or optical (e.g., infrared) receivers and transmitters. The specific design and implementation of the communication subsystem 1024 can depend on the communication network(s) over which a mobile device is intended to operate. For example, architecture 1000 can include communication subsystems 1024 designed to operate over a GSM network, a GPRS network, an EDGE network, a Wi-Fi™ network and a Bluetooth™ network. In particular, the wireless communication subsystems 1024 can include hosting protocols, such that the mobile device can be configured as a base station for other wireless devices.

Audio subsystem 1026 can be coupled to a speaker 1028 and a microphone 1030 to facilitate voice-enabled functions, such as voice recognition, voice replication, digital recording and telephony functions. Audio subsystem 1026 can be configured to receive voice commands from the user.

I/O subsystem 1040 can include touch surface controller 1042 and/or other input controller(s) 1044. Touch surface controller 1042 can be coupled to a touch surface 1046. Touch surface 1046 and touch surface controller 1042 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 1046. Touch surface 1046 can include, for example, a touch screen or the digital crown of a smart watch. I/O subsystem 1040 can include a haptic engine or device for providing haptic feedback (e.g., vibration) in response to commands from processor 1004. In an embodiment, touch surface 1046 can be a pressure-sensitive surface.

Other input controller(s) 1044 can be coupled to other input/control devices 1048, such as one or more buttons, rocker switches, thumb-wheel, infrared port and USB port. The one or more buttons (not shown) can include an up/down button for volume control of speaker 1028 and/or microphone 1030. Touch surface 1046 or other controllers 1044 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 1046; and a pressing of the button for a second duration that is longer than the first duration may turn power to the mobile device on or off. The user may be able to customize a functionality of one or more of the buttons. The touch surface 1046 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 1002 can be coupled to memory 1050. Memory 1050 can include high-speed random access memory and/or non-volatile memory, such as one or more magnetic disk storage devices, one or more optical storage devices and/or flash memory (e.g., NAND, NOR). Memory 1050 can store operating system 1052, such as the iOS operating system developed by Apple Inc. of Cupertino, Calif. Operating system 1052 may include instructions for handling basic system services and for performing hardware dependent tasks. In some implementations, operating system 1052 can include a kernel (e.g., UNIX kernel).

Memory 1050 may also store communication instructions 1054 to facilitate communicating with one or more additional devices, one or more computers and/or one or more servers, such as, for example, instructions for implementing a software stack for wired or wireless communications with other devices. Memory 1050 may include graphical user interface instructions 1056 to facilitate graphic user interface processing; sensor processing instructions 1058 to facilitate sensor-related processing and functions; phone instructions 1060 to facilitate phone-related processes and functions; electronic messaging instructions 1062 to facilitate electronic-messaging related processes and functions; web browsing instructions 1064 to facilitate web browsing-related processes and functions; media processing instructions 1066 to facilitate media processing-related processes and functions; GNSS/Location instructions 1068 to facilitate generic GNSS and location-related processes and instructions; and heart rate instructions 1070 to facilitate hear rate measurements. Memory 1050 further includes activity application (e.g., a fitness application) instructions for performing the features and processes described in reference to FIGS. 1-9.

Each of the above identified instructions and applications can correspond to a set of instructions for performing one or more functions described above. These instructions need not be implemented as separate software programs, procedures, or modules. Memory 1050 can include additional instructions or fewer instructions. Furthermore, various functions of the mobile device may be implemented in hardware and/or in software, including in one or more signal processing and/or application specific integrated circuits.

The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language (e.g., SWIFT, Objective-C, C#, Java), including compiled or interpreted languages, and it can be deployed in any form, including as a stand-alone program or as a module, component, subroutine, a browser-based web application, or other unit suitable for use in a computing environment.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
obtaining, using one or more processors, acceleration and rotation rate in a body frame of a wearable device worn on a limb of a user engaged in a physical activity;
transforming, using the one or more processors, the acceleration and rotation rate into an inertial frame;
determining, using the one or more processors, vertical acceleration in the inertial frame, rotation rate magnitude and vertical acceleration in the inertial frame due to the user's limb rotation;
determining, using the one or more processors, a work rate (WR) based caloric expenditure for the user based on the vertical acceleration, rotation rate magnitude and a correlation coefficient that measures a correlation between the vertical acceleration in the inertial frame and the vertical acceleration in inertial frame due to the user's limb rotation;
obtaining, using the one or more processors, heart rate (HR) data from a heart rate sensor of the wearable device;
determining, using the one or more processors, an HR based caloric expenditure based on the HR data; and
fusing, the WR based caloric expenditure with the HR based caloric expenditure to get a fused caloric expenditure.

2. The method of claim 1, further comprising:
displaying, on a display of the wearable device the fused caloric expenditure, or sending by the wearable device the fused caloric expenditure to another device, or storing on wearable device or another device, the fused caloric expenditure.

3. The method of claim 1, further comprising:
obtaining, using the one or more processors, at least one of the user's weight and the user's height; and
determining, using a WR model, the WR based caloric expenditure for the user based on the vertical acceleration, rotation rate magnitude, a correlation coefficient that measures a correlation between the vertical acceleration in the inertial frame and the vertical acceleration in inertial frame due to the user's limb rotation, the user's weight and the user's height.

4. The method of claim 1, further comprising:
compensating the HR data with a HR drift factor that is computed based on an amount of time the user is engaged in a physical activity of a specified intensity.

5. The method of claim 1, further comprising:
obtaining, using the one or more processors, the user's age and the user's estimated maximal oxygen uptake; and
determining, using the one or more processors, an HR based caloric expenditure based on the HR data, the user's age and the user's estimated maximal oxygen uptake.

6. The method of claim 1, wherein fusing, the WR based caloric expenditure with the HR based caloric expenditure to get a fused caloric expenditure, further comprises:
averaging the WR based caloric expenditure and the HR based caloric expenditure.

7. The method of claim 1, wherein the physical activity is dancing.

8. The method of claim 1, further comprising:
obtaining a plurality of reference vertical acceleration data points in the inertial frame that are associated with human body motion for a particular physical activity;
subtracting the reference vertical acceleration from the vertical acceleration in the inertial frame that was measured at the user's wrist to obtain vertical acceleration in the inertial frame due to rotation of the user's limb; and
determining a correlation coefficient representing the correlation using a linear regression model where the independent variable is the vertical acceleration in the inertial frame due to rotation of the user's limb and the dependent variable is the rotation rate of the user's limb in the inertial frame.

9. The method of claim 1, wherein the limb is the user's arm and the wearable device is worn on the user's wrist.

10. The method of claim 1, wherein the WR and HR caloric expenditures are based on metabolic equivalent of tasks (MET) values.

11. A system comprising:
motion sensors;
one or more processors;
memory storing instructions that when executed by the one or more processors, cause the one or more processors to perform operations comprising:
obtaining, from the motion sensors, acceleration and rotation rate in a body frame of a wearable device worn on a limb of a user engaged in a physical activity;
transforming the acceleration and rotation rate into an inertial frame;
determining vertical acceleration in the inertial frame, rotation rate magnitude and vertical acceleration in the inertial frame due to the user's limb rotation;
determining a work rate (WR) based caloric expenditure for the user based on the vertical acceleration, rotation rate magnitude and a correlation coefficient that measures a correlation between the vertical acceleration in the inertial frame and the vertical acceleration in inertial frame due to the user's limb rotation;
obtaining heart rate (HR) data from a heart rate sensor of the wearable device;
determining an HR based caloric expenditure based on the HR data; and
fusing, the WR based caloric expenditure with the HR based caloric expenditure to get a fused caloric expenditure.

12. The system of claim 11, further comprising:
a display configured to display the fused caloric expenditure; and
a wireless transmitter configured to send the fused caloric expenditure to another device.

13. The system of claim 11, the operations further comprising:
obtaining at least one of the user's weight and the user's height; and
determining, using a WR model, the WR based caloric expenditure for the user based on the vertical acceleration, rotation rate magnitude, a correlation coefficient that measures a correlation between the vertical acceleration in the inertial frame and the vertical acceleration in inertial frame due to the user's limb rotation, the user's weight and the user's height.

14. The system of claim 11, the operations further comprising:
compensating the HR data with a HR drift factor that is computed based on an amount of time the user is engaged in a physical activity of a specified intensity.

15. The system of claim 11, the operations further comprising:
obtaining the user's age and the user's estimated maximal oxygen uptake; and
determining an HR based caloric expenditure based on the HR data, the user's age and the user's estimated maximal oxygen uptake.

16. The system of claim 11, the operations further comprising:
averaging the WR based caloric expenditure and the HR based caloric expenditure.

17. The system of claim 11, wherein the physical activity is dancing.

18. The system of claim 11, the operations further comprising:
obtaining a plurality of reference vertical acceleration data points in the inertial frame that are associated with human body motion for a particular physical activity;
subtracting the reference vertical acceleration from the vertical acceleration in the inertial frame that was measured at the user's wrist to obtain vertical acceleration in the inertial frame due to rotation of the user's limb; and
determining a correlation coefficient representing the correlation using a linear regression model where the independent variable is the vertical acceleration in the inertial frame due to rotation of the user's limb and the dependent variable is the rotation rate of the user's limb in the inertial frame.

19. The system of claim 11, wherein the limb is the user's arm and the wearable device is worn on the user's wrist.

20. The system of claim 11, wherein the WR and HR caloric expenditures are based on metabolic equivalent of tasks (MET) values.
